(19) United States
(12) Patent Application Publication
(10) Pub. No.: US 2011/0104265 A1
(43) Pub. Date: May 5, 2011

(54) COMPOSITIONS AND METHODS OF TARGETED NANOFORMULATIONS IN THE MANAGEMENT OF OSTEOPOROSIS

(76) Inventors: Shaker A. Mousa, Wynantskill, NY (US); Mohammed H. Qari, Jeddah (SA); Mohammed S. Ardawi, Jeddah (SA)

(21) Appl. No.: 12/912,902

(22) Filed: Oct. 27, 2010

Related U.S. Application Data
(60) Provisional application No. 61/279,961, filed on Oct. 29, 2009.

(51) Int. Cl. A61K 9/48; A61K 38/18; A61K 31/351; A61K 31/56; A61K 38/23; A61K 38/11; A61K 38/29; A61K 38/22; A61K 31/4045; A61P 19/10

(52) U.S. Cl. 424/451; 514/8.8; 514/460; 514/169; 514/11.9; 514/11.6; 514/11.8; 514/5.8; 514/419

(57) ABSTRACT

A composition and method for treating a bone condition of an animal. The compostion includes nanoparticles; a targeted moiety covalently bonded to an outer surface of each nanoparticle; and osteoblast stimulating molecules encapsulated within each nanoparticle. The method for treating a bone condition includes introducing the composition into the animal.

### CROSS-REFERENCES TO RELATED APPLICATIONS

This patent application claims priority to U.S. Provisional No. 61/279,961, filed on Oct. 29, 2009, which is incorporated herein by reference in its entirety.

### FIELD OF THE INVENTION

The present invention pertains to methods and compositions for prevention and/or treatment of a bone condition such as osteoporosis and bone fracture.

### BACKGROUND OF THE INVENTION

Osteoporosis is a disease with a very wide distribution all over the world, it leads to bones being porous and fragile, the complications of this disease include fractures and delayed healing. The burdens of such global problem are immense to the health care providers due to the increasing cost of treatment and prevention, and the increasing morbidity and mortality. Many international and local studies are published including those from Saudi Arabia and Arabian Gulf states all agrees on the increasing risks of osteoporosis. Fractures and its complications, and the need to have more emphasis on research that leads to improvement of the strategies of prevention and treatment.

Although neither calcium nor vitamin D has been shown to prevent osteoporosis in postmenopausal women alone, the combination does. Both calcium and vitamin D are commonly used in the treatment of osteoporosis. The estrogens and raloxifene both prevent bone loss in postmenopausal women, and the estrogens probably also decrease the risk of first fracture. There is good evidence that raloxifene prevents further fractures in postmenopausal women who have already had fractures and some evidence that estrogen does as well. Calcitonin increases bone mineral density in early postmenopausal women and men with idiopathic osteoporosis, and also reduces the risk of new fractures in osteoporotic women. The bisphosphonate alendronate prevents bone loss and reduces fractures in healthy and osteoporotic postmenopausal women, and in osteoporotic men. Risedronate is more potent and has fewer upper gastrointestinal side effects than alendronate, and reduces the incidence of fractures in osteoporotic women. Intermittent use of the potent bisphosphonate zoledronate also increases bone mineral density and may become an alternative in the prevention and treatment of osteoporosis. All of the agents discussed above prevent bone resorption, whereas teriparatide increases bone formation and is effective in the treatment of osteoporotic women and men. In the treatment of secondary osteoporosis associated with the use of glucocorticoids to treat inflammation or prevent rejection after transplantation, the bisphosphonates are effective. The agents that have undergone some clinical trialing as new or alternative drugs for the treatment of osteoporosis include tibolone, new SERMs, androgens, growth hormone, insulin-like growth factor-1 and stontium ranelate. The targets/drugs that are being developed to inhibit bone resorption include the OPG/ RANKL/RANK system, cathepsin K inhibitors, vitronectin receptor antagonists, estren, the interleukin-6 and gp130 system, cytokines and growth factors. New drugs/targets to promote bone formation include the commonly used lipid-lowering statins and the calcilytic release of PTH.

There is a need for improved treatment of osteoporosis.

### SUMMARY OF THE INVENTION

The present invention provides a composition, comprising: nanoparticles; a targeted moiety covalently bonded to an outer surface of each nanoparticle; and osteoblast stimulating molecules encapsulated within each nanoparticle.

The present invention provides a method for treating a bone condition of an animal, said method comprising: treating the bone condition of the animal, said treating comprising introducing the composition of the present invention into the animal.

### DETAILED DESCRIPTION OF THE INVENTION

Nanotechnology based novel drug-carrying systems that specifically delivered to osteoporotic bone are utilized in the present invention. Some of these novel drug carrying systems distribute pharmaceutical agents locally to quickly increase bone mass where a balanced inhibition of overactive osteoclast is achieved while stimulating suppressed osteoblast in osteoporosis. All available approaches are associated with major adverse effects and single sided efficacy either on osteoclast or osteoblast leading to imbalanced effects on bone turnover rate and unhealthy bone. The present invention provides targeted nano-delivery systems to osteoporotic bone utilizing covalently bonded bisphosphonate and/or high affinity αcβ3 ligand on the nano surface and encapsulating osteoblast stimulating molecules such as estrogen-related compounds, calcitonin, parathyroid hormone (PTH), statins, and teriparatide.

The novel targeted delivery systems of the present invention focus on the prolonged release of bioactive agents, specifically bone morphogenetic proteins (BMPs), and in particular BMP-2 or 4, osteoblast stimulating molecules such as estrogen-related compounds, calcitonin, PTH, statins, and teriparatide to efficiently regenerate healthy bone. Nanoparticle formulations utilizing inorganic biodegradable nanomaterials (including ceramic materials such as hydroxyapatite and hyaluronan), chitosan linked with hyaluronan and collagen were functionalized with bioactive chemicals (such as potent αvβ3 ligand, namely SM256 or XT199 with a Ki of 1-40 nM, and model compounds expected to increase bone cell function) or bisphosphonate. Such bioactive groups are placed on the outer surface of the nanoparticles using various techniques resulting in covalent chemical bonding. The outer coating of the embedded nanoparticle systems have different biodegradation rates for the controlled release of embedded bioactive agents to the target site. In this manner, ceramic nanoparticles drug delivery systems are available for the management of osteoporosis.

Nanotechnology based novel drug-carrying systems that specifically attach to osteoporotic bone are utilized. Moreover, some of these novel drug carrying systems distribute pharmaceutical agents locally to quickly increase bone mass. These efforts focus on the prolonged release of bioactive agents specifically bone morphogenetic protein-2 (BMP-2) to efficiently regenerate enough bone for the patient to return to a normal active lifestyle. Particularly, inorganic biodegradable nanomaterials (including ceramic materials such as hydroxyapatite or HA) were functionalized in a study with bioactive chemicals (such as potent avb3 ligand, a model compound known to increase bone cell function). Such bioactive groups were placed on the outer surface of the nanoparticle systems using various techniques resulting in covalent chemical attachment. The outer coating of the embedded nanoparticle systems were also created to have different biodegradation rates for the controlled release of embedded bioactive agents to the target site. In this manner, ceramic nanoparticles drug delivery systems were developed for osteoporosis.

### EXAMPLES

### Example 1

The study is a prospective controlled trial on an animal model using the New Zealand rabbit, in which osteoporosis is induced through the removal of the ovaries.

A composition of the present invention may be administered in any desired and effective manner: as compositions for oral ingestion, or for parenteral or other administration in any appropriate manner such as intraperitoneal, subcutaneous, intratumoral, topical, intradermal, inhalation, intranasal, rectal, vaginal, sublingual, intramuscular, intravenous, intra-arterial, intrathecal, or intralymphatic. Regardless of the route of administration selected, the composition may be formulated into pharmaceutically-acceptable dosage forms by conventional methods known to those of ordinary skill in the art (e.g., see: Remington's Pharmaceutical Sciences, Mack Publishing Co., Easton, Pa.). Pharmaceutical carriers are well known in the art (e.g., see: Remington's Pharmaceutical Sciences cited above and The National Formulary, American Pharmaceutical Association, Washington, D.C.) and include sugars (e.g., lactose, sucrose, mannitol, and sorbitol), starches, cellulose preparations, calcium phosphates (e.g., dicalcium phosphate, tricalcium phosphate and calcium hydrogenphosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly[orthoesters], and poly[anhydrides]), elastomeric matrices, liposomes, microspheres, oils (e.g., corn, germ, olive, castor, sesame, cottonseed, and groundnut), cocoa butter, waxes, paraffins, silicones, talc, silicylate, and the like. Suitable carriers used included in the composition of the present invention should be compatible with the other ingredients of the composition. Carriers suitable for a selected dosage form and intended route of administration are well known in the art, and acceptable carriers for a chosen composition, dosage form and method of administration can be determined using ordinary skill in the art.

The composition of the present invention may, optionally, contain one or more additional agents commonly used in pharmaceutical compositions. These agents are well known in the art and include but are not limited to (1) fillers or extenders, such as starches, lactose, sucrose, glucose, mannitol, silicic acid or the like; (2) binders, such as carboxymethylcellulose, alginates, gelatin, polyvinyl pyrrolidone, hydroxypropylmethyl cellulose, sucrose, acacia or the like; (3) humectants, such as glycerol or the like; (4) disintegrating agents, such as agar-agar, calcium carbonate, potato or tapioca starch, alginic acid, certain silicates, sodium starch glycolate, cross-linked sodium carboxymethyl cellulose, sodium carbonate or the like; (5) solution retarding agents, such as paraffin or the like; (6) absorption accelerators, such as quaternary ammonium compounds or the like; (7) wetting agents, such as acetyl alcohol, glycerol monostearate or the like; (8) absorbents, such as kaolin, bentonite clay or the like; (9) lubricants, such as talc, calcium stearate, magnesium stearate, solid polyethylene glycols, sodium lauryl sulfate or the like; (10) suspending agents, such as ethoxylated isostearyl alcohols, polyoxyethylene sorbitol and sorbitan esters, microcrystalline cellulose, aluminum metahydroxide, bentonite, agar-agar, tragacanth or the like; (11) buffering agents; (12), excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, polyamide powder or the like; (13) inert diluents, such as water, other solvents or the like; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, waxes or the like; (18) opacifying agents; (19) adjuvants; (20) emulsifying and suspending agents; (21), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, fatty acid esters of sorbitan or the like; (22) propellants, such as chlorofluorohydrocarbons or the like and volatile unsubstituted hydrocarbons, such as butane, propane or the like; (23) antioxidants; (24) agents which render the formulation isotonic with the blood of the intended recipient, such as sugars, sodium chloride or the like; (25) thickening agents; (26) coating materials, such as lecithin or the like; and (27) sweetening, flavoring, coloring, perfuming and preservative agents. Each such ingredient or material should be compatible with the other ingredients of the formulation. Agents suitable for a selected dosage form and intended route of administration are well known in the art, and acceptable ingredients and materials, dosage form and method of administration may be readily determined by those of ordinary skill in the art.

A composition in accordance with the present invention that are suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations can be prepared by methods well known in the art.

In one embodiment, the composition of the present invention comprises nanoparticles; a targeted moiety covalently bonded to an outer surface of each nanoparticle; and osteoblast stimulating molecules encapsulated within each nanoparticle.

In one embodiment, the the nanoparticles are selected from the group consisting of an inorganic biodegradable nanomaterial, chitosan, chitosan cross linked with fatty acid or bile acid, chitosan linked with hyaluronan, collagen, hydrogel, poly(lactic-co-glycolic acid) (PLGA), chitosan, chitosan cross linked with fatty acid or bile acid, and combinations thereof.

In one embodiment, the nanoparticles comprise the inorganic biodegradable nanomaterial, wherein the inorganic biodegradable nanomaterial is a ceramic material.

In one embodiment, the ceramic material is selected from the group consisting of hydroxyapatite, hyaluronan, and a combination thereof.

In one embodiment, the targeted moiety is selected from the group consisting of bisphosphonate, αvβ3 ligand, and a combination thereof.

In one embodiment, the osteoblast stimulating molecules are selected from the group consisting of bone morphogenetic proteins (BMPs), estrogen-related compounds, calcitonin, parathyroid hormone (PTH), statins, teriparatide, leptin, PPAR-γ suppressor, SIRT-1 inducers, cathepsin K inhibitors, melatonin, and combinations thereof.

The present invention provides a method for treating a bone condition of an animal, wherein the method comprises: treating the bone condition of the animal, said treating comprising introducing the composition of the present invention into the animal.

In one embodiment, the animal is a human being.

In one embodiment, the animal is a non-human species of animal.

In one embodiment, the bone condition being treated is osteoporosis.

In one embodiment, the bone condition being treated is a bone fracture.

In one embodiment, said treating the bone condition results in bone regeneration in the animal.

In one embodiment, said introducing the composition comprises introducing the composition into the animal via a delivery selected from the group consisting of an oral delivery, an intranasal delivery, a subcutaneous delivery, an intravenous delivery, an intraperiteoneal delivery, a topical delivery, and combinations thereof.

While particular embodiments of the present invention have been described herein for purposes of illustration, many modifications and changes will become apparent to those skilled in the art. Accordingly, the appended claims are intended to encompass all such modifications and changes as fall within the true spirit and scope of this invention.

What is claimed is:

1. A composition, comprising:
nanoparticles;
a targeted moiety covalently bonded to an outer surface of each nanoparticle; and
osteoblast stimulating molecules encapsulated within each nanoparticle.

2. The composition of claim 1, wherein the nanoparticles are selected from the group consisting of an inorganic biodegradable nanomaterial, chitosan, chitosan cross linked with fatty acid or bile acid, chitosan linked with hyaluronan, collagen, hydrogel, poly(lactic-co-glycolic acid) (PLGA), chitosan, chitosan cross linked with fatty acid or bile acid, and combinations thereof.

3. The composition of claim 2, wherein the nanoparticles comprise the inorganic biodegradable nanomaterial, and wherein the inorganic biodegradable nanomaterial is a ceramic material.

4. The composition of claim 3, wherein the ceramic material is selected from the group consisting of hydroxyapatite, hyaluronan, and a combination thereof.

5. The composition of claim 1, wherein the targeted moiety is selected from the group consisting of bisphosphonate, αvβ3 ligand, and a combination thereof.

6. The composition of claim 5, wherein the targeted moiety comprises bisphosphonate.

7. The composition of claim 5, wherein the targeted moiety comprises αvβ3 ligand.

8. The composition of claim 1, wherein the osteoblast stimulating molecules are selected from the group consisting of bone morphogenetic proteins (BMPs), estrogen-related compounds, calcitonin, oxytocin, parathyroid hormone (PTH), statins, teriparatide, leptin, PPAR-γ suppressor, SIRT-1 inducers, cathepsin K inhibitors, melatonin, and combinations thereof.

9. The composition of claim 8, wherein the osteoblast stimulating molecules comprise said BMPs.

10. The composition of claim 8, wherein the osteoblast stimulating molecules comprise said estrogen-related compounds.

11. The composition of claim 8, wherein the osteoblast stimulating molecules comprise said PTH.

12. A composition formation method, comprising:
forming the composition of claim 1.

13. The method of claim 12, wherein the targeted moiety is biodegradable, and wherein said forming comprises selecting the targeted moiety that has a rate of biodegration for controlling release of the osteoblast stimulating molecules from the nanoparticles.

14. A method for treating a bone condition of an animal, said method comprising:
treating the bone condition of the animal, said treating comprising introducing the composition of claim 1 into the animal.

15. The method of claim 14, wherein the animal is a human being.

16. The method of claim 14, wherein the animal is a non-human species of animal.

17. The method of claim 14, wherein the bone condition being treated is osteoporosis.

18. The method of claim 14, wherein the bone condition being treated is a bone fracture.

19. The method of claim 14, wherein said treating the bone condition results in bone regeneration in the animal.

20. The method of claim 14, wherein said introducing the composition comprises introducing the composition into the animal via a delivery selected from the group consisting of an oral delivery, an intranasal delivery, a subcutaneous delivery, an intravenous delivery, an intraperiteoneal delivery, a topical delivery, and combinations thereof.
